(19) United States
(12) Plant Patent Application Publication
(10) Pub. No.: US 2002/0178476 P1
(43) Pub. Date: Nov. 28, 2002

(54) Chrysanthemum plant named 'Mother Teresa'

(76) Inventor: Subodh Kumar Datta, Lucknow (IN)

Correspondence Address: Foley & Lardner, Suite 500, 3000 K Street, N.W., Washington, DC, 20007-5109, US

(21) Appl. No.: 09/821,100

(22) Filed: Mar. 30, 2001

(51) Int. Cl. A01H 5/00

(52) U.S. Cl. PLT/294

(57) ABSTRACT

The present invention relates to a novel hybrid plant of Chrysanthenum morifolium Ramat christened as ‘Mother Teresa’ and having dwarf, bushy compact round shaped: profuse blooming habit, producing white Anemone type flowers 1th distinct white ray florets and creamish white disc florets, green foliage with fewv white streaks and requiring nether ‘Pinching’ nor ‘Staking’.

### FIELD OF THE INVENTION

The present invention relates to a new ornamental chrysanthemum plant christened as ‘Mother Teresa’

### BACKGROUND OF THE INVENTION

The present invenrion relates to a now and distinct variety of ‘Chrysanthemum morifolium’ Ramat., a member of the Chrysanthemum genus, which has been developed using the open pollinated seedling method. Chrysanthenum morifolium is a popular flower crop of world-wide commercial importance. Chrysanthemum varieties have earned tremendous popularity as an ornamental flower for garden, as cut flowers for interior decoration and loose flower. Demand of new and novel varieties of Chrysanthemum are increasing day-by-day in the world floriculture trade.

Chrysanthemum can be propagated both by seed and vegetative means. Cross breeding and selective breedng helped in developing large number of new varieties. The genotype ‘Mother Teresa’ has evolved through seedling selections of open pollinations among specific group of chrysanthemum. The new genotype evolved in this invention represents such an improvement combining novel flower colour and shape nd plant stature. The new variety offers the floriculturists/nurserymen an improved attactive novel ornamental plant which can be commerically cultivated.

### SUMMARY OF THE INVENTION

The present invention provides a new and disdinct hybrid Chrysanthemum morifolium variety christened as ‘Mother Teresa’ characterized by profise blooming habit, dwarf bush, white Anemone type mini plant habit.

### DETAILED DESCRIPTION OF THE INVENTION

The invention provides a new, novel and distinct hybrid Chtysanthemum morifolium plant christened as ‘Mother Teresa’, having the following combination of characters:

(a) Green foliage with few white streaks,

(b) White Anemone type flower;

(c) Dwarf, bushy, compact round shaped, profuse bloomng plant, suitable for mini culture;

(d) Pigment composition of petals being distinct from other existing varieties, and

(f) True-to-type performance under different environments.

The new variety of this invention is a Chrysanthemum morifolium genotype, christened as ‘Mother Teresa’. This planit has been developed through planned breeding programmes conducted at National Botanical Research Institute, Lucknow, India with an aim to develop a new hybrid variety of Chrysanthemum morifolium. For this purpose existing mini. varieties of chrysanthemum i.e. Haldighati and Swama Singar were grown very closely in the field in August 1989 at Lucknow, India facilitating pollnation among themselves. The seeds borne on existing cultivars (Haldighati and Swarna Singar) were collected in January 1990 and were sown in February 1990. One of the plants in the field was a dwarf plant that exhibited Anemone type flowers and profuse blooming This plant was given number DWS 13 and was selected for further observation and evaluation.

Considering the attractive flower colour and shape and plant growth habit, it was asexally reproduced through suckers to maintain clonal purity. The selected clone DWS 13 was grown both in beds and 10″ earthen pots alongwith existing mini varieties (Haldighati and Swarna Singar)for five successive generations from 1990 upto 1995. The clone DWS 13 maintained its flower colour and shape and plant growth habit in all the generations. This plant was found to be different from all other chrysanthemum plants in colour of flowers, shape and pigment conmposition of petals.

In the subsequent year (1996), the genotype DWS 13 was evaluated in both replicated field and pot tnal alongwith existing parents to study the flower colour and shape and plant growth habit. The clone DWS 12 bred true to type and proved very attractive over the existing varieties in quality of bloom including colour, shape and type of bloom. Chrysanthemum cultivation (large and small flowered cultivars) require lot of cultural operations including ‘Pinching’ and ‘Staking’. For small flowered Chrysanthemum apical shoot tips are cut (half to one inch) to give proper shape to the plant. This operation is called “Pinching” and “Staking” is necessary to keep piants erect and to maintain proper shape of plant and bloom. These operations are expensive and time consuming.

National Botanical Research Institute, Lucknow, India has already developed ‘Mini Chrysanthemum’ which is unique of its kind. It requires neither ‘Pinching’ nor ‘Staking’. It is a unique geineic strain with dwarf, bushy, compact round shaped, profuse blooming habit. Number of existing mini varieties and their colour is very limited at present, Efforts were rmade to develop new flower colour under an on going improvement research programme.

The new clone DWS 13, selected from open pollinated seedling, is a product of this Research Programme and has been Christened ‘Mother Teresa’,

### EVIDENCE OF UNIFORMITY AND STABILITY

The genotype ‘Mother Teresa’ has remained stable and uniform for its morphological characters and showed consistency in performance for various vegetative and floral quality attributes during its evaluation and vegetative multiplication from 1990 until 1995.

No variation was detected in morphological characters especially in the flower clour characters through vegetative multiplication durnng large scale propagation, testing and field trial upto 1997.

### BRIEF DESCRIPTION OF THE ACCOMPANYING DRAWINGS, AND IN THESE DRAWINGS:

FIG. 1 represents colour photograph of pot grown plant of ‘Mother Teresa’ depicting flower argemenents, shape and colour,

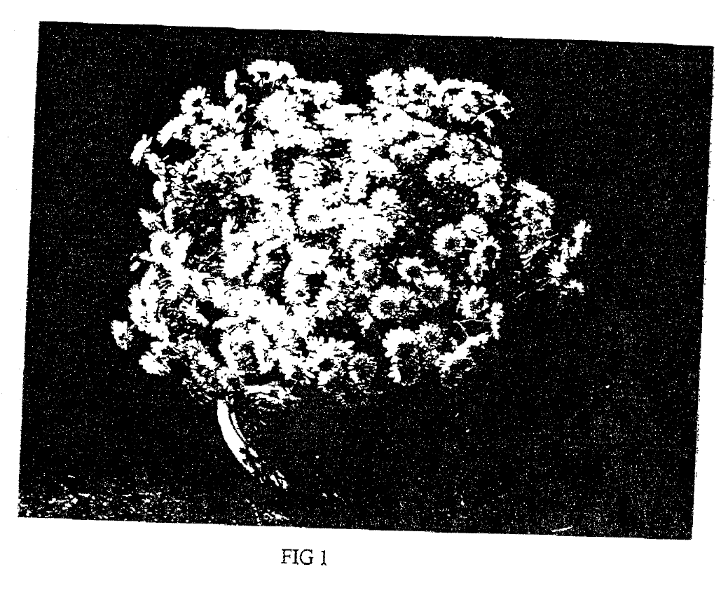

FIG. 2 represents close-up colour photograph of ‘Mother Teresa’ depicting anemone type arrangement of ray florets (white) and disc florets (slightly creamish white).

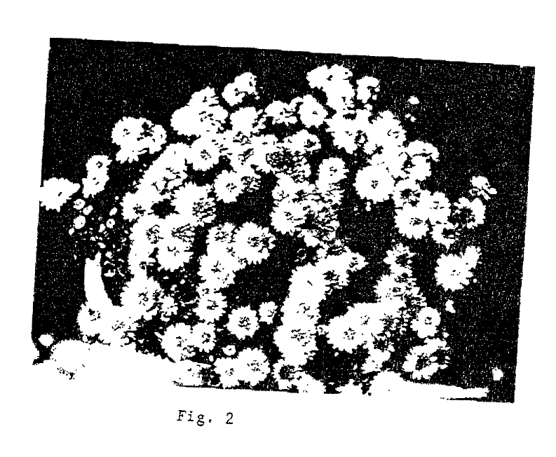

### STATEMENT OF DISTINCTION

The genotype ‘Mother Teresa’ possesses small, thin green leaves with very smal white streaks on it white Anemone type flower, dwarf bushy, compact round shaped, profuse blooming habit.

The genotype ‘Mother Teresa’ is distinct in regeneration potential in second and subsequent generations in morphological and flower characters.

Repeated petal extracts from different plants of Mother Teresa showed the same peaks for pigments in spectrophotometer.

### PIGMENT ANALYSIS

The main difference between new hybrid ‘Mother Teresa and two existing parental varieties (Haldighati and Swarna Singer) was in flower colour and shape. Therefore, flower pigment of three genotypes were analyzed to establish the relatedness among them.

For detection of pigment composition florets (1 gm) of three genotypes were extracted in methanol containing 1% HCI and centrifuged. The supernatant was taken into volumetric flask and volume was made upto 50 ml. The extract was scanned from 200 to 700 nm wavelenght in spectrophotometer The details of compound in different genotypes are as followd

| | No. of Peak | | |
|---|---|---|---|
| Genotype | (Compound) | Peak Height | Wave length |
| Mother Teresa | 1 | 0.768 | 204.3 |
| | 2 | 0.256 | 268.2 |
| Haldighati | 1 | 0.450 | 192.1 |
| | 2 | 1.419 | 204.5 |
| | 3 | 0.887 | 328.0 |
| | 4 | 0.093 | 415.4 |
| | 5 | 0.122 | 434.8 |
| | 6 | 0.109 | 462.0 |
| Swarna Singar | 1 | 0.609 | 193.1 |
| | 2 | 2.079 | 206.6 |
| | 3 | 0.882 | 248.6 |
| | 4 | 0.780 | 268.1 |
| | 5 | 0.177 | 330.4 |
| | 6 | 0.192 | 434.3 |
| | 7 | 0.167 | 462.1 |

Pigment analysis clearly indicate that genotype ‘Mother Teresa’ was very distinct in pigment composition from two other parental genotypes both qualitatively and quantitatively.

### OBJECTIVE DESCRIPTION OF TEH VARIETY “MOTHER TERESA” (DWS 13 )

The following is an objective desription of the new variety:

1. Genus: Chrysanthemum

2. Species: morifoium Ramant.

3. Family: Compositae

4. Common name: English: Chrysanthemum; Local names: Sanskrit—Sevanti; Hindi—Guldaudi; Bengali—Chadramallika; Marathi—Shevati; Tamil—Akkarakkam; Telgu—Chamunti; Punjabi—Gondi, Bagaura; Urdu—Guledawoodi

5. Plant Height: About 2

6. Growth Habit: Dwarf, busy, compact

7. Stem: Quadrangular, green

8. Leaf: small, thin, green with very small white streaks:

Texture.—Thin.

Surface.—Smooth (rarely few hairs).

Shape.—Oval.

Tip.—Acute.

Size.—Broad.

Length.—3.2 cm.

Width.—1.8 cm.

Petiole length.—04 cm.

9. Inflorescence; Capitullum

Ray florets.—White, 1-2 whorl

Disc forets.—Compact, slightly creamish white, disc very promnent

10. Flower type: Anemone

The salient features of ‘Mother Teresa’ and two other existing varieties are as follows.

| | Varieties | | |
|---|---|---|---|
| Characters | Mother Teresa | Haldighati | Swrana Singar |
| Leaf length (cm) | 3.2 | 2.6 | 2.4 |
| Leaf width (cm) | 1.8 | 1.4 | 1.5 |
| Petiole length (cm) | 0.4 | 1.1 | 1.0 |
| Plant height (cm) | 22 | 27.5 | 14.0 |
| Floret/head | 34 | 32 | 76 |
| Floret length (cm) | 1.2 | 1.2 | 1.3 |
| Floret width (cm) | 0.5 | 0.4 | 1.4 |
| Bloom type | 1-2 whorl ray | 1-3 whorl ray | Double Korean |
| | floret, flat, | floret, flat, | |
| | disc prominent, | disc prominent, | |
| | Anemone type | Decorative type | |
| Floret colour | White ray floret, | Bright yellow | Yellow group |
| | light yellowish | Yellow group | (12A) |
| | White group | (9A) | FAN 1 |
| | (158A) | FAN 1 | |
| | FAN 4 | | |
| No. of pigment | 2 | 6 | 7 |

The floret colour which is the main novelty of ornamentals was identified in the present varieties according to R.H.S. Colour Chart published by The Royal Horticultural Society London in association with the Flower Council of Holanld, 1966.

1. A novel hybrid plant of Chysanthemum morifolium Ramat christened as ‘Mother Teresa’ and having the following characteristics;
(a) dwarf, bushy, compact round shaped, profuse blooming, habit,
(b) requiring neither ‘Pinching’ nor ‘Staking’,
(c) producing white Anemone type flowers with distinct white ray florets and creamish white disc florets ((158A) (FAN 4;)—(RHS), and
(d) green foliage with few white streaks.
